(19) United States
(12) Patent Application Publication
(10) Pub. No.: US 2021/0386891 A1
(43) Pub. Date: Dec. 16, 2021

(54) Disinfecting System

(71) Applicant: Vivienne Campbell, Riverside, CT (US)

(72) Inventor: Vivienne Campbell, Riverside, CT (US)

(21) Appl. No.: 17/034,038

(22) Filed: Sep. 28, 2020

Related U.S. Application Data
(60) Provisional application No. 63/037,703, filed on Jun. 11, 2020.

(51) Int. Cl. A61L 2/22; A61L 2/24; A61L 2/26

(52) U.S. Cl. CPC A61L 2/22; A61L 2/24; A61L 2202/14; A61L 2202/15; A61L 2/26

(57) ABSTRACT

A disinfectant dispensing system for automatically cleaning a high-traffic surface. The automatic disinfectant dispensing system is configured to spray a disinfecting or sanitizing solution on a high-traffic surface for a predetermined time long enough to clean the surface after the surface has been touched and potentially contaminated. A liquid disinfecting or sanitizing solution is retained in a fluid storage component. The solution is dispensed to a plurality of fluid dispensing components via distribution tubing in fluid communication with the fluid storage component. Each of the fluid dispensing components is positioned near a high-traffic surface, such as a doorknob. A motion sensing element in each fluid dispensing components detects the presence of an external object, such as a hand, and activates a fluid dispersal element to dispense the disinfecting or sanitizing solution a period of time after the motion is no longer detected.

### CROSS-REFERENCE TO RELATED APPLICATION

The present application claims priority to, and the benefit of, U.S. Provisional Application No. 63/037,703, which was filed on Jun. 11, 2020 and is incorporated herein by reference in its entirety.

### BACKGROUND

The present invention generally relates to a disinfecting system, and more specifically to a disinfectant dispersal apparatus system for automatically cleaning a high-traffic surface. Accordingly, the present specification makes specific reference thereto. However, it is to be appreciated that aspects of the present invention are also equally amenable to other like applications, devices and methods of manufacture.

High-traffic public and private areas have surfaces such as doorknobs, toilet flush handles, elevator keypads, and the like that may easily become contaminated with germs, viruses, bacteria, and other pathogens. Homeowners, maintenance staff, and guests may lack the means to properly clean each area after a person touches it. Or, they may simply not realize that they have contaminated these surfaces. People with illnesses can easily spread their germs, bacteria, and other contaminates to the surface where they are spread to others when they touch these contaminated surfaces. Standard cleaning methods may be time consuming, not frequent enough, and ultimately ineffective.

Germs, viruses, bacteria, microbes, and other pathogens are tiny living things found everywhere in nature and are too small to be seen by the naked eye. These organisms are constantly present and persist on a wide variety of surfaces including clothing and human skin. Microbes live in water, soil, and in the air. A pathogen is a micro-organism that has the potential to cause disease. People carry these pathogens into their homes unknowingly because they lack a proper means to sanitize or disinfect their persons before entering their house. This puts other members of the house at greater risk of infection as they interact with the person introducing the pathogens into the house. When uninfected people touch a contaminated surface, they can easily introduce these pathogens into mucus membranes that exist in the eyes, nose, and mouth. These infections can lead to serious illness or even death.

Viruses are a common cause of infectious diseases. Transmission can easily occur by indirect transmission through contaminated surfaces. Disinfectants and sanitizing agents are used to clean contaminated surfaces as one of the more frequently implemented measures to control transmission of pathogens in indoor environments. Disinfectants and sanitizers have proven effective at reducing the overall number of the disease causing microorganisms that cause illness on a surface. Regular and routine cleaning is necessary to prohibit the continued spread of these microorganisms. While cleaning is simple to perform, its efficacy is questionable, and in many cases is subject to time between applications, chemical performance and human error. Additionally, the cleaning effect is short lived, being limited to the point when recontamination of the surface occurs. Once a surface is contaminated again, the pathogens will continue to survive until the area is disinfected again.

Viruses such as COVID-19, which is a virus caused by a coronavirus called SARS-CoV-2, the flu, and the common cold are easily spread. Older adults and people who have severe underlying medical conditions like heart or lung disease or diabetes seem to be at higher risk for developing more serious complications from these illnesses. They are thought to spread mainly from person to person, mainly through respiratory droplets produced when an infected person coughs or sneezes and the droplets land on a surface. There is no way to tell if the surface is contaminated visually.

Applying soap and water mechanically to a surface is an effective method of cleaning. However, this method is not easily automated. Disinfection and sanitization are processes of decontamination. Sanitizers are designed to reduce the overall number of the microorganisms on a surface. Sanitizers have a more broad and immediate use against a wide range of microbes to reduce the overall number of microbes on a surface, thus reducing the chances of illness. A disinfectant typically has a stronger effect than a sanitizer on some parthenogenic microbes on a surface. Disinfectants normally have a more focused and specific use against certain bacteria, viruses, and disease. Disinfection eliminates most pathogens but not necessarily all types of microbes. Disinfectants also require a “dwell time” to be effective, meaning they must remain on an inanimate surface for a certain period of time to be effective against the specific microbe(s). Both disinfectants and sanitizers are optimal for automated application as a spray application of the agent can remain on the surface for the “dwell time” before it dries out optimizing the sanitizing or disinfecting process.

In this manner, the improved disinfecting system of the present invention accomplishes all of the forgoing objectives, thereby providing an easy solution to decrease the transmission of disease. A primary feature of the present invention disinfectant dispersal system for cleaning a high traffic surface. Finally, the improved disinfectant dispersal system of the present invention is capable of facilitating the automatically cleaning of a high-traffic surface, preventing the spread of germs, viruses, and bacteria.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises an automatic dispensing system for dispensing a fluid such as a cleaning solution. The automatic dispensing system may be placed or positioned in locations near high-traffic areas with frequently touched surfaces, such as a doorknob, an elevator button, or a flush handle. The cleaning solution may be a disinfectant, a sanitizing solution, or a mixture thereof that can be dispensed on the frequently touched surface as an aerosolized spray a period of time after the frequently touched surface was last touched.

The automatic dispensing system comprises a fluid storage component, a distribution component, and at least one fluid dispensing component. The fluid storage component comprises an interior reservoir for retaining the cleaning solution. The interior reservoir may retain the cleaning solution directly, or a bag or other container of the cleaning solution that may be inserted within. The fluid storage component is attachable to a surface, such as a wall or door, adjacent to the frequently touched surface to be cleaned.

The distribution component is in fluid communication with the fluid storage component and comprises an outlet tube and a plurality of dispensing component tubes. The outlet tube engages the fluid storage component through a reservoir connection end that penetrates the interior reservoir of the fluid storage component. The plurality of dispensing component tubes branch out distally from the opposite end of the outlet tube. Each of the plurality of dispensing component tubes comprise a dispensing component outlet that enters the corresponding fluid dispensing component. Each of the plurality of dispensing component tubes may further comprise a valve controllable by the corresponding fluid dispensing component for releasing the cleaning solution into the fluid dispensing component.

Each fluid dispensing component comprises a motion sensing element and a fluid dispersal element. The motion sensing element may comprise a sensing circuit and a circuit board in electrical communication with the sensing circuit. The sensing circuit is configured to actively or passively detect the presence of an external object, such as a hand, as it engages the frequently touched surface. The fluid dispersal element comprises a plurality of orifices for dispersing the cleaning solution as a spray or aerosol. Each fluid dispensing component may further comprises a spray angle adjusting element for directing the spray or aerosol. The cleaning solution is dispersed after the motion sensing element stops detecting the presence of the external object.

Each fluid dispensing component may further comprise a pumping element and a timer. Both the pumping element and the timer are in electrical communication with the sensing circuit. Each fluid dispensing component further comprises a battery for powering the fluid dispensing component. The pumping element enables fluid dispersal element to disperse a quantity of the cleaning solution a predetermined period of time set by the timer after the motion sensing element stops detecting the presence of an external object and opens the valve.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of an automatic dispensing system of the present invention for dispensing a cleaning solution in accordance with the disclosed architecture.

FIG. 2 illustrates a perspective view one potential embodiment of a fluid storage component of the automatic dispensing system of the present invention in accordance with the disclosed architecture.

FIG. 3 illustrates a side view of one potential embodiment of the fluid storage component of the automatic dispensing system of the present invention in accordance with the disclosed architecture.

FIG. 4 illustrates a perspective view of one potential embodiment of a distribution component of the automatic dispensing system of the present invention in accordance with the disclosed architecture.

FIG. 5 illustrates a perspective view of one potential embodiment of a plurality of fluid dispensing components of the automatic dispensing system of the present invention in accordance with the disclosed architecture.

FIG. 6 illustrates an overhead view of one potential embodiment of one of the fluid dispensing components of the automatic dispensing system of the present invention in accordance with the disclosed architecture.

FIG. 7 illustrates a perspective view of one potential embodiment of one of the fluid dispensing components of the automatic dispensing system of the present invention dispensing a cleaning solution on a high-traffic area surface in accordance with the disclosed architecture.

FIG. 8 illustrates a rear perspective view of one potential embodiment of a spray angle adjusting element of one of the fluid dispensing components of the automatic dispensing system of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They do not intend as an exhaustive description of the invention or do not limit the scope of the invention. Additionally, an illustrated embodiment need not have all the aspects or advantages shown. Thus, in other embodiments, any of the features described herein from different embodiments may be combined.

The present invention, in one exemplary embodiment, provides users with a disinfecting system capable of automatically spraying a sanitizer or disinfectant onto high-traffic areas such as door handles, elevator buttons, and the like. The system features a refillable main dispersing unit capable of storing a disinfecting or sanitizing liquid. The system also includes at least one motion sensor activated dispenser that can be installed over the high-traffic surface via adhesive, suction cups, or the like. The at least one motion sensor activated dispenser may be connected to the refillable main dispersing unit by a small rubber or plastic hose for fluid dispensing.

A device features a main storage unit for retaining the sanitizer that is connected to the at least one dispenser via the tubing that is activated by a motion sensor. The system may be mounted to a wall surface via a fastening system, such as suction, adhesive, or mechanical fasteners. Each motion sensor may be generally circular or geometric in shape with several dispensing holes on the base. A backside of each dispenser may have a small tab or other adjusting mechanism for adjusting the angle of the dispenser in relation to the wall surface.

The system may be installed in a bathroom, near a door, in an elevator, or in other high-traffic areas. The system may be filled with a sanitizing or disinfecting agent and activated. The motion sensors detect when a person operates or touches the high-traffic area (e.g. a doorknob). Once the user's hand is removed from the high-traffic area, the disinfecting or sanitizing solution is sprayed onto the surface. The system ensures that these areas are automatically cleaned after use preventing the spread of germs, viruses, and bacteria.

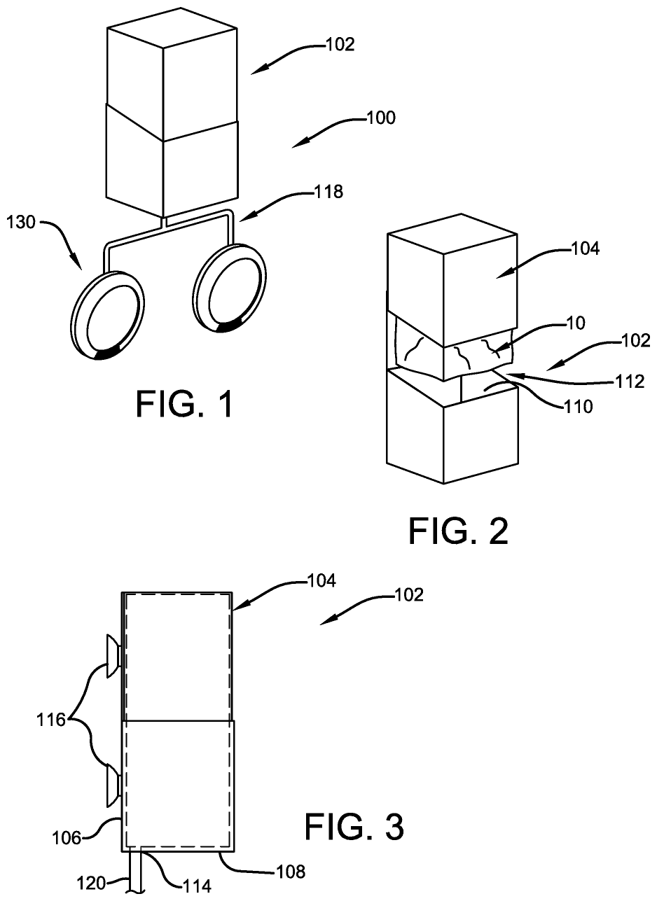

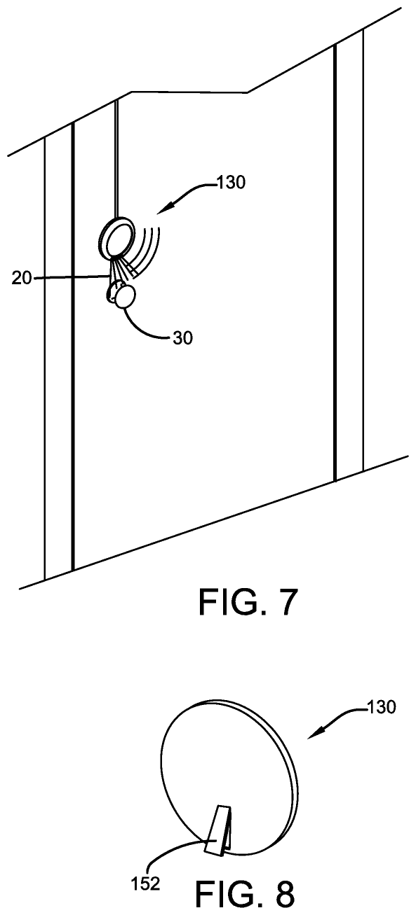

Referring initially to the drawings, FIG. 1 illustrates an automatic dispensing system 100. The automatic dispensing system 100 is configured for use in retaining and dispensing a fluid, such as a cleaning solution 10 as illustrated in FIG. 2. The automatic dispensing system may be placed in locations near high-traffic areas with frequently touched surfaces 30, such as a doorknob, an elevator button, or a flush handle as illustrated in FIG. 7. The cleaning solution 10 may be a disinfectant, a sanitizing solution, or a mixture thereof that can be dispensed on the frequently touched surface 30 as an aerosolized, fine droplet spray, or mist 20 a period of time after the frequently touched surface was last touched.

The automatic dispensing system 100 comprises a fluid storage component 102, a distribution component 118, and at least one fluid dispensing component 130. As illustrated in FIGS. 2 and 3, the fluid storage component 102 comprises a housing 104 and an interior reservoir 110 for retaining the cleaning solution 10. The housing 104 comprises a back 106 and a bottom 108. The interior reservoir 110 may retain the cleaning solution 10 directly, or a bag or other container of the cleaning solution 10 that may be inserted or loaded within through an opening 112 in the housing 102.

As illustrated in FIG. 7, the fluid storage component 102 is attachable to a surface, such as a wall or door, adjacent to the frequently touched surface 30 to be cleaned via a mounting element 116. The mounting element 116 may be a bracket, suction element, adhesive, mechanical fastener, or the like, typically attached to the back 106 of the housing 102. Alternatively, the mounting element 116 could be positioned anywhere on an external surface of the housing as desired, such as a hanger (not shown) attached to a top of the housing 104. The fluid storage component 102 further comprises an outlet 114 in the bottom 108 or other lower portion of the housing 104 for engaging the distribution component 118.

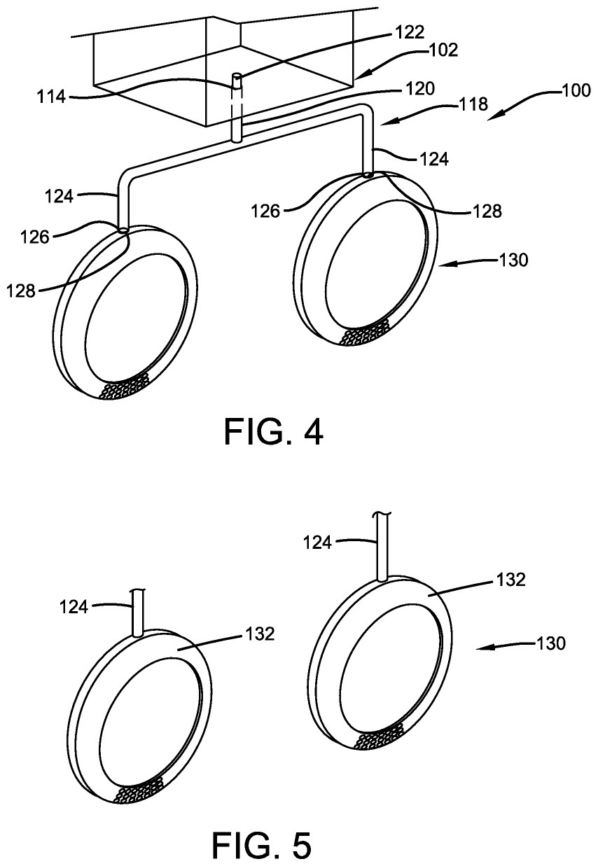

As illustrated in FIG. 4, the distribution component 118 is in fluid communication with the fluid storage component 102. The distribution component 118 comprises an outlet tube 120 and a plurality of dispensing component tubes 124. The outlet tube 120 and the plurality of dispensing component tubes 124 are typically manufactured from plastic, silicone or rubber tubing or hose. The outlet tube 120 engages the fluid storage component 102 through a reservoir connection end 122. The reservoir connection end 122 penetrates the housing 104 into the interior reservoir 110 of the fluid storage component 102.

Each of the plurality of dispensing component tubes 124 branch out distally from an end of the outlet tube 120 opposite the reservoir connecting end 122. Each of the plurality of dispensing component tubes 124 comprise a dispensing component outlet 126 that penetrates or enters the corresponding fluid dispensing component 130. Each of the plurality of dispensing component tubes 124 may further comprise a valve 128 controllable by the corresponding fluid dispensing component 130 for releasing the cleaning solution 10 into the fluid dispensing component 130. Alternatively, the valve 128 could be located anywhere within the automatic dispensing system 100

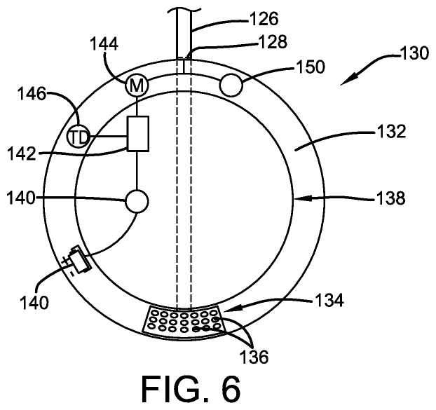

As illustrated in FIGS. 5 and 6, the at least one fluid dispensing component 130 may be a plurality of fluid dispensing components 130 each supplied by the corresponding dispensing component tube 124. Each fluid dispensing component 130 comprises a housing 132. The housing is typically a circular, oval, or geometrically shaped plastic case. Each fluid dispensing component 130 further comprises a motion sensing element 138 and a fluid dispersal element 134. The fluid dispersal element 134 comprises a plurality of orifices 136 or nozzles that penetrate the housing 132 and are configured to disperse the fluid 10 in an aerosolized spray or mist after the motion sensing element 138 stops detecting the presence of an external object.

The motion sensing element 138 may comprise a sensing circuit 140 and a circuit board 142 in electrical communication with the sensing circuit 140. The sensing circuit 140 is configured to actively or passively detect the presence of an external object, such as a hand, as it engages the frequently touched surface. The motion sensing element 138 may be an active or passive motion sensor. The motion sensing element 138 may activate the valve 128 to release the cleaning solution 10 into the fluid dispersal element 134 for dispersion onto the frequently touched surface 30.

Each fluid dispensing component 130 further comprises a pumping element 144 and a timer 146. Both the pumping element 144 and the timer 146 are in electrical communication with the sensing circuit 140 and the circuit board 142. The pumping element 144 may be a motor electrically connected to the circuit board 142. The pumping element 144 enables fluid dispersal element 134 to disperse a quantity of the cleaning solution 10 after the motion sensing element 138 stops detecting the presence of the external object. The timer 146 delays the dispersal of a quantity of the cleaning solution 10 for a predetermined period of time after the motion sensing element stops detecting the presence of an external object. The timer 146 may permit a user to adjust the predetermined period of time as desired. In addition to a manual rubber button, the timer 146, the pumping element 144, and the motion sensing element 138, individually or in combination, may control activation and deactivation of the valve 128 to release the cleaning solution 10.

Each fluid dispensing component 130 further comprises a battery 148 for powering each fluid dispensing component 130. As illustrated in FIG. 8, each fluid dispensing component 130 further comprises a spray angle adjusting element 152. Each spray angle adjusting element 152 may be a tab, or similar structure, that extends out of a back of the housing 132 laterally to change the angle of the plurality of orifices 136 of the fluid dispersal element 134 allowing the installer to direct the spray of mist 20 of the cleaning solution 10. Additionally, each fluid dispensing component 130 may comprise a manual activation element 150 in electrical communication with the pumping element 144 for manually activating the fluid dispensing component 130 as desired.

In a further embodiment of the invention, the automatic dispensing system 100 may further comprise a fill/refill indicator line to show when the cleaning solution 10 is full and/or needs replenished (e.g., a 10% low), and a manual rubberized button with an elbow pictured on the front center of the dispensary unit/units to allow manual backup activation for instant dispensing of the solution 10.

It is contemplated that the automatic dispensing system 100 constructed in accordance with the present invention will be tailored and adjusted by those of ordinary skill in the art to accommodate various levels of performance demand imparted during actual use. Accordingly, while this invention has been described by reference to certain specific embodiments and examples, it will be understood that this invention is capable of further modifications. This application is, therefore, intended to cover any variations, uses or adaptations of the invention following the general principles thereof, and including such departures from the present disclosure as come within known or customary practice in the art to which this invention pertains and fall within the limits of the appended claims.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An automatic dispensing system comprising:
a fluid storage component;
a distribution component in fluid communication with the fluid storage component; and
at least one fluid dispensing component supplied by the distribution component, the at least one fluid dispensing component comprising a motion sensing element and a fluid dispersal element; and
wherein the fluid dispersal element disperses a fluid after the motion sensing element stops detecting the presence of an external object.

2. The automatic dispensing system of claim 1, wherein the fluid is a disinfectant solution.

3. The automatic dispensing system of claim 1, wherein the fluid is a sanitizing solution.

4. The automatic dispensing system of claim 1, wherein the fluid dispersal element disperses the fluid in an aerosolized spray.

5. The automatic dispensing system of claim 1, wherein the fluid dispersal element disperses the fluid a predetermined period of time after the motion sensing element stops detecting the presence of an external object.

6. The automatic dispensing system of claim 1, wherein the motion sensing element is an active motion sensor.

7. The automatic dispensing system of claim 1, wherein the automatic dispensing system is battery powered.

8. An automatic dispensing system for dispensing a cleaning solution comprising:
a fluid storage component for retaining the cleaning solution;
a distribution component in fluid communication with the fluid storage component; and
a plurality of fluid dispensing components supplied by the distribution component, each fluid dispensing component comprising a motion sensing element, a pumping element, and a fluid dispersal element; and
wherein the pumping element enables fluid dispersal element to disperse a quantity of the cleaning solution after the motion sensing element stops detecting the presence of an external object.

9. The automatic dispensing system of claim 8, wherein the distribution component comprises a valve activated by the motion sensing element to release the cleaning solution into the fluid dispersal element.

10. The automatic dispensing system of claim 8, wherein the motion sensing element comprises a sensing circuit and a circuit board in electrical communication with the pumping element.

11. The automatic dispensing system of claim 8, wherein each fluid dispensing component further comprises a timer in electrical communication with the motion sensing element.

12. The automatic dispensing system of claim 11, wherein each timer delays the dispersal of the quantity of the cleaning solution a predetermined period of time after the motion sensing element stops detecting the presence of the external object.

13. The automatic dispensing system of claim 12, wherein the predetermined period of time is adjustable.

14. The automatic dispensing system of claim 8, wherein each fluid dispensing component is battery powered.

15. The automatic dispensing system of claim 8, wherein each fluid dispensing component further comprises a spray angle adjusting element.

16. An automatic dispensing system for dispensing a cleaning solution comprising:
a fluid storage component for retaining the cleaning solution;
a distribution component in fluid communication with the fluid storage component; and
a plurality of fluid dispensing components supplied by the distribution component, each fluid dispensing component comprising a motion sensing element, a pumping element, a timer, and a fluid dispersal element; and
wherein the pumping element enables fluid dispersal element to disperse a quantity of the cleaning solution a predetermined period of time set by the timer after the motion sensing element stops detecting the presence of an external object.

17. The automatic dispensing system of claim 16, wherein the motion sensing element comprises a sensing circuit and a circuit board in electrical communication with the timer and the pumping element.

18. The automatic dispensing system of claim 16, wherein the distribution component comprises a valve activated by the timer to release the cleaning solution into the fluid dispersal element.

19. The automatic dispensing system of claim 16, wherein each fluid dispensing component further comprises a spray angle adjusting element.

20. The automatic dispensing system of claim 16, wherein the fluid dispersal element is manually activatable.
